(19) United States
(12) Patent Application Publication
(10) Pub. No.: US 2005/0118254 A1
(43) Pub. Date: Jun. 2, 2005

(54) Microemulsion preconcentrate

(76) Inventors: Jae Mook Choi, Kwangju (KR); Eun Kyung Jeon, Yongin-city (KR); Jae Kyoung Ko, Incheon-city (KR); Young Hwan Park, Kangrung-city (KR); Myoung Ki Baek, Seoul (KR); Su Geun Yang, Yongin-city (KR); Eun Ju Jeong, Jincheon-gun (KR)

Correspondence Address: CANTOR COLBURN, LLP, 55 GRIFFIN ROAD SOUTH, BLOOMFIELD, CT, 06002, US

(21) Appl. No.: 10/500,230

(22) PCT Filed: Dec. 26, 2002
(86) PCT No.: PCT/KR02/02443

(30) Foreign Application Priority Data
Dec. 27, 2001 (KR) 10-2001-0085994

(51) Int. Cl. A61K 38/13; A61K 31/7072; A61K 31/704; A61K 31/56; A61K 31/195

(52) U.S. Cl. 424/451; 514/11; 514/254.07; 514/355; 514/171; 514/291; 514/571; 514/449; 514/49; 514/34; 514/561; 514/411

(57) ABSTRACT

A microemulsion preconcentrate is provided, which comprises an active component, an oil, a surfactant, and a hydrophilic solvent selected from the group consisting of propylene glycol diacetate, propylene glycol monoacetate, and salts of the forgoing materials.

### TECHNICAL FIELD

The present invention relates to a microemulsion preconcentrate.

### BACKGROUND ART

Microemulsions are used as solubilizing formulation for hydrophobic drugs poorly soluble in water. Oil-in-water (O/W) microemulsions are difficult to commercially produce because its external phase is water and its stability during shelf-life does not reach a desired level. For this reason, drug-containing capsulated microemulsion preconcentrates consisting of a hydrophilic phase, a lipophilic phase, and a surfactant have often been used. After oral administration, the capsulated microemulsion preconcentrate is disintegrated and dissolved by a gastric juice to form microemulsion.

Examples of microemulsion preconcentrates include Sandimmun Neoral™ carrying cyclosporin, a widely known hydrophobic drug, which is disclosed in EP520949A1 (Novartis), Cardus marianus extract or Silibin, which is disclosed in U.S. 2001/005726AA and an oral microemulsion composition containing biphenyl dimethyl dicarboxylate as an active component, which is disclosed in Korean Laid-Open Publication No. 1998-083257.

However, the microemulsion preconcentrates disclosed in the above patents are only for carrying hydrophobic drugs, not for hydrophilic drugs or protein drugs, and thus have limited applications.

The manufacture of drugs with such microemulsion preconcentrates is limited by the choice of their hydrophilic phase. For example, propylene glycol, polyethylene glycol, or ethanol, if used for the hydrophilic phase, may vaporize or may interact with and be absorbed into a gelatin shell of a soft capsule over time during capsulation, thereby changing the original composition of the microemulsion, and eventually leading to precipitation and separation of the drug. In particular, ethanol may vaporize completely over time.

Soft capsules lose their shape due to a reaction of their gelatin shell with the hydrophilic phase of the microemulsion during capsulation, and the contents leak through gaps in a seam, thereby lowering the yield. During processes of drying and aging soft capsules, an irreversible solvent substitution between the moisture in the gelatin shell and the hydrophilic phase of the microemulsion, and migration of other components, occur, thereby greatly changing the original composition of the hydrophilic phase in the microemulsion. As a result, the drug is separated to destroy the microemulsion system. These adverse phenomena continue to appear through the shelf-life, making it difficult to mass produce and mass market drug microemulsions.

### DISCLOSURE OF THE INVENTION

The present invention provides a microemulsion preconcentrate capable of delivering hydrophilic and protein drugs as well as hydrophobic drugs, and having no interaction with a gelatin shell during capsulation to thus secure the stability of the product.

According to one aspect of the invention, there is provided a microemulsion preconcentrate comprising: an active component; an oil; a surfactant; and a hydrophilic solvent selected from the group consisting of propylene glycol diacetate, propylene glycol monoacetate, and salts of the forgoing materials.

In the microemulsion preconcentrate according to the present invention, preferably, the ratio by weight of the sum of oil, hydrophilic solvent, and surfactant to the active component is 0.5-10. It is preferable that the ratio by weight of oil, hydrophilic solvent, and surfactant is 0.5-60: 0.5-60:0.5-80. More preferably, the ratio by weight of oil, hydrophilic solvent, and surfactant is 5-30: 5-30: 5-60.

The microemulsion preconcentrate according to the present invention may further comprise a pharmaceutically acceptable additive. The pharmaceutically acceptable additive may be at least one selected from the group consisting of an antioxidant, a thickening agent, a preservative, and a flavoring agent.

The present invention provides an oral pharmaceutical preparation comprising the microemulsion preconcentrate. The oral pharmaceutical preparation may be any dosage forms for example, soft capsules, gelatin-sealed hard capsules, or liquid.

### BRIEF DESCRIPTION OF THE DRAWINGS

FIG. 1 represents the granularity distribution result of a microemulsion composition diluted with water from a cyclosporin microemulsion preconcentrate formulated according to Example 1-a; and

FIG. 2 shows photographs of soft capsules: one filled with composition (B) according to example 1-a according to the present invention and the other filled with conventional composition (A), wherein both capsules were exposed to the air for 30 days after capsulation.

### BEST MODE FOR CARRYING OUT THE INVENTION

Hereinafter, the present invention will be described in detail.

A microemulsion preconcentrate according to the present invention basically comprises a base compositon including a hydrophilic solvent, an oil, and a surfactant, and a pharmaceutically active component. The pharmaceutical active component is mixed with and dissolved in the base composition to yield the microemulsion preconcentrate. The hydrophilic solvent is propylene glycol diacetate, propylene glycol monoacetate, or a salt of the forgoing materials. These hydrophilic solvents may be mixed in any combination.

Propylene glycol diacetate, amphipathic solvent for both hydrophobic drugs, such as cyclosporin, and hydrophilic drugs, has a molecular weight of about 160 and a boiling point of 186° C., so it is less volatile at room temperature and less reactive with a gelatin capsule shell as compared with conventionally used propylene glycol or ethanol. Accordingly, propylene glycol diacetate is suitable for the hydrophilic solvent.

In the microemulsion preconcentrate according to the present invention, preferably, the ratio by weight of a base compositon including a hydrophilic solvent, an oil, and a surfactant to the active component is 0.5-10. It is preferable that the ratio by weight of oil, hydrophilic solvent, and surfactant is 0.5-60: 0.5-60:0.5-80. More preferably, the ratio by weight of oil, hydrophilic solvent, and surfactant is 5-30: 5-30: 5-60.

The microemulsion preconcentrate according to the present invention may further include pharmaceutically acceptable additives, such as an antioxidant, a thickening agent, a preservative, a dissolution regulator, a flavoring agent, a coloring agent, and the like. For example, antioxidants may include tocopherols and salts thereof; thickening agents may include polymers, such as hydroxypropyl cellulose, hydroxypropylmethylcellulose, methylcellulose, and Eudragit™; flavoring agents may include apple, pineapple flavors, and the like; and preservatives may include benzoic acid.

Pharmaceutically acceptable active components for the microemulsion preconcentrate according to the present invention may include, but are not limited to: anti-inflammatory agents and anodynes, such as piroxicam, ketorolac, ketopropen, acetaminophen, aceclofenac, naproxen, gabapentin, and the like; anti-hypertensive drugs, such as amlodipine, felodipine, enalapril, isosorbide dinitrate, terazocine, carvedilol, nifedipine, captopril, and the like; antifungal agents, such as itraconazole, fluconazole, ketoconazole, and the like; anticancer drugs, such as fluorouracil, paclitaxel, adriamycin, and the like; steroid drugs, such as estradiol, progestin, testosterone, and the like; erectile dysfunction drugs, such as alprostadil; anti-Alzheimer drugs, such as donepezil, rivastigmine, physostigmine, adrenol, and the like; anti-osteoporesis drugs, such as alendronate; immunizing agents, such as cyclosporin, tacrolimus, and the like; antiemetic agents, such as ondansetron, scopolamine, meclizine, and the like; tranquilizers, such as fluoxetine, venlafaxine, and the like; and pharmaceutically acceptable salts of the forgoing drugs.

The microemulsion preconcentrate according to the present invention may include, as the active component, not only the above-listed synthetic drugs, peptide, and hormonal drugs, but also recombinant protein drugs, such as human insulin, human growth hormones, erythropoietin, human epidermal cell growth factor, and the like.

A suitable surfactant for the microemulsion preconcentrate according to the present invention may be at least one of, but is not limited to, polyoxyethylene glycolated natural or hydrogenated vegetable oils, polyoxyethylene sorbitan fatty acid esters, polyoxyethylene fatty acid esters, polyoxyethylene-polyoxypropylene copolymers, dioctylsuccinate, dioctylsodium sulfosuccinate, di-[α-ethylhexyl]-succinate or sodium laurylsulfate, phospholipids, phospholipid derivatives, polyethylene glycol mono- and di-fatty acid esters, bile acids, bile salts, trans-esterification products of natural vegetable oil triglycerides and polyalkylene polyols, esterification products of caprylic or capric acid with glycerol, sorbitan fatty acid esters, pentaerythrite fatty acid esters and pentaerythritol fatty acid esters, polyalkylene glycol ethers, polyethylene glycol 660 12-hydroxy stearate, tocopheryl polyethylene glycol 1000 succinate, and cholesterols and derivatives thereof.

Polyoxyethylene glycolated natural or hydrogenated vegetable oils, reaction products of natural or hydrogenated vegetable oils and ethylene glycol, are commercially available under the trade names of “Cremophor RH 40”, “Cremophor EL”, etc.

Polyoxyethylene sorbitan fatty acid esters are commercially available under the trade name “Tween”. Tween 20 and Tween 80 are preferred as surfactants for the microemulsion preconcentrate according to the present invention.

Polyoxyethylene fatty acid esters are commercially available under the trade names of “Myrj” and “Briji.”

Polyoxyethlene-polyoxypropylene copolymers are commercially available under the trade names of “Poloxamer” and “Pluronic.”

Examples of polyethylene glycol mono- and di-fatty acid esters include polyethylene glycol dicaprylate, polyethylene glycol dilaurate, polyethylene glycol hydroxystearate, polyethylene glycol isostearate, polyethylene glycol laurate, polyethylene glycol ricinolate, and polyethylene glycol stearate.

A representative example of bile acids and bile salts is sodium taurocholate.

Trans-esterification products of natural vegetable oil triglycerides and polyalkylene polyols are commercially available under the trade name of “Labrafil”. Labrafil M 1944 CS and “Labrasol” are preferred as surfactants for the microemulsion preconcentrate according to the present invention.

Esterification products of caprylic or capric acid with glycerol are commercially available under the trade name of “Imwitor™”.

Examples of sorbitan fatty acid esters include sorbitan-monolauryl ester, sorbitan-monopalmityl ester, sorbitan-monostearyl ester, sorbitan-tristearyl ester, sorbitan-monooleyl ester, and sorbitan-trioleyl ester, which are commercially available under the trade name of “Span”.

The above-listed surfactants may be used separately alone or in a combination of at least two of the surfactants, with the use of at least two surfactants being preferred.

An example of oil that can be used for the microemulsion preconcentrate according to the present invention includes, but is not limited to, at least one selected from the group consisting of vegetable oils, animal oils, esterification products of vegetable fatty acids, unsaturated long chain fatty acids, esterification products of unsaturated long chain fatty acids, tocopherols, and tocopherol derivatives.

Examples of vegetable oils for the microemulsion preconcentrate according to the present invention include corn oil, borage oil, sesame oil, primrose oil, peanut oil, olive oil, and poppy seed oil. Examples of animal oils include squalenes and omega-3 fatty acids consisting of eicosapentaenoic acid (EPA) and docosahexaenoic acid (DHA).

Examples of the esterfication products of vegetable oil fatty acids include fatty acid triglycerides, fatty acid mono- and di-glycerides, fatty acid mono- and di-acetylated monoglycerides. Examples of unsaturated long chain fatty acids include linoleic acid and oleic acid.

Examples of the esterification products of unsaturated long chain fatty acids include ethyl linoleate, ethyl oleate, and ethyl myristate. Examples of tocopherols and derivatives thereof include tocopherol acetates and dl-alpha-tocopherol.

The above-listed oils may be used separately alone or in a combination of at least two of the oils.

The microemulsion preconcentrate is used for preparing an oral pharmaceutical preparation by conventional methods known in the field. The pharmaceutical preparation may have diverse dosage forms, example soft capsules, gelatin-sealed hard capsules, or liquid. For example, a pharmaceutically active component is dissolved in a hydrophilic solvent under mild heating. An oil and a surfactant are added into the mixture and homogeneously mixed, and if necessary, a pharmaceutically acceptable additive is added into the mixture. The final composition is processed into soft capsules using a soft-capsule manufacturing machine.

The present invention will be described in greater detail with reference to the following examples. The following examples are for illustrative purposes and are not intended to limit the scope of the invention.

### EXAMPLE 1

### Manufacture of Cyclosporin Microemulsion Preconcentrate and Soft Capsules

100 g of cyclosporin, an active component, was dissolved in a hydrophilic solvent containing 10 g of propylene glycol monoacetate and 150 g of propylene glycol diacetate under heating with stirring. 50 g of Peceol, 60 g of Capmul, and 130 g of Labrafac as oils, and 350 g of Cremphor RH 40 and 200 g of Labrasol as surfactatnts were added into the solution and mixed by stirring to yield a homogeneous microemulsion preconcentrate. The resulting microemulsion preconcentrate was poured into a soft capsule manufacturing machine and shaped into soft capsules according to general procedures widely used in the field. Each capsule contained 100 mg of cyclosporin.

Soft capsules of different microemulsion preconcentrate compositions, as shown in Table 1 below, were manufactured for Examples 1-a, 1-b, and 1 c, using the same method as described above.

TABLE 1
units: grams

| | | | Exam- | Exam- | Exam |
|---|---|---|---|---|---|
| | | | ple | ple | ple |
| | Component | Example 1 | 1-a | 1-b | 1-c |
| Hydrophilic | Propyleneglycol | 150 | 225 | 120 | 250 |
| solvent | diacetate | | | | |
| | Propyleneglycol | 100 | — | — | — |
| | monoacetate | | | | |
| Surfactant | Cremophor RH 40 | 350 | 450 | 400 | 450 |
| | Tween 20 | — | 120 | — | 50 |
| | Labrasol | 200 | — | 150 | — |
| Oil | Peceol | 50 | 125 | — | — |
| | Capmul MCM | 60 | — | 120 | — |
| | Labrafac CC | 130 | 150 | 150 | — |
| | Tocopherol acetate | — | — | — | 300 |
| Active | Cyclosporin | 100 | 100 | 100 | 100 |
| Component | | | | | |

### EXAMPLES 2 to 5

### Manufacture of Microemulsion Preconcentrates of Various Drugs and Soft Capsules

Microemulsion preconcentrates of various drugs, having the compositions shown in Table 2 below, were prepared, and soft capsules of the microemulsion preconcentrates were manufactured, using the same methods as described in Example 1. Each capsule contained an effective dose of the active component required for a particular therapeutic effect.

TABLE 2
units: grams

| | | Exam- | Exam- | Exam- | Exam- |
|---|---|---|---|---|---|
| | | ple | ple | ple | ple |
| | Component | 2 | 3 | 4 | 5 |
| Hydrophilic | Propyleneglycol | 250 | — | 150 | 180 |
| solvent | diacetate | | | | |
| | Propyleneglycol | — | 200 | 70 | — |
| | monoacetate | | | | |
| Surfactant | Cremophor RH 40 | 400 | 350 | 330 | — |
| | Poloxamer 124 | 100 | — | 120 | 350 |
| | Labrafil | — | 150 | — | 150 |
| Oil | Mivacet | 50 | 120 | — | — |
| | Ethyl linoleate | 60 | — | 120 | — |
| | Labrafac CC | 140 | 160 | 150 | 250 |
| Active | Ondansetron | 100 | — | — | — |
| component | Gabapentin | — | 100 | — | — |
| | Alendronate | — | — | 100 | — |
| | Venlafaxine | — | — | — | 100 |

### EXAMPLES 6 to 8

### Manufacture of Microemulsion Preconcentrates of Various Drugs and Soft Capsules

Microemulsion preconcentrates of various drugs, having the compositions shown in Table 3 below, were prepared, and soft capsules of the microemulsion preconcentrates were manufactured, using the same methods as described in Eexample 1. Each capsule contained an effective dose of the active component required for a particular therapeutic effect.

TABLE 3
units: grams

| | Component | Example 6 | Example 7 | Example 8 |
|---|---|---|---|---|
| Hydrophilic | Propyleneglycol | 150 | 225 | 120 |
| solvent | diacetate | | | |
| | Propyleneglycol | 100 | — | — |
| | monoacetate | | | |
| Surfactant | Poloxamer 124 | 400 | 450 | 450 |
| | Tween 80 | 100 | 120 | — |
| | Labrasol | — | — | 100 |
| | Yolk Lecithin | — | — | 150 |
| Oil | Ethyl myristate | 50 | 50 | — |
| | Capmul MCM | 50 | — | 120 |
| | Labrafac CC | 130 | 150 | — |
| | Lipiodol | — | — | 200 |
| Active | Itraconazole | 100 | — | — |
| component | Prostaglandin | — | 100 | — |
| | Paclitaxel | — | — | 100 |

### EXAMPLES 9 to 11

### Manufacture of Microemulsion Preconcentrates of Various Drugs and Soft Capsules

Microemulsion preconcentrates of various drugs, having the compositions shown in Table 4 below, were prepared, and soft capsules of the microemulsion preconcentrates were manufactured, using the same methods as described in Example 1. Each capsule contained an effective dose of the active component required for a particular therapeutic effect.

TABLE 4
units: grams

| | Component | Example 9 | Example 10 | Example 11 |
|---|---|---|---|---|
| Hydrophilic | Propyleneglycol | 150 | 225 | 120 |
| solvent | diacetate | | | |
| | Propyleneglycol | — | — | 50 |
| | monoacetate | | | |
| Surfactant | Solutol HS 15 | 200 | 150 | 450 |
| | Labrasol | — | — | 50 |
| | Yolk Lecithin | — | — | 150 |
| | Capmul MCM | 50 | — | 120 |
| | Labrafac CC | — | 150 | — |
| | Linoleic acid | — | — | 50 |
| | Lactic acid | 100 | — | 50 |
| Active | Insulin | 100 | — | — |
| Component | Human EGF | — | 100 | — |
| | hormone | | | |
| | Interferon | — | — | 100 |

### EXPERIMENTAL EXAMPLE 1

### Granularity Distribution Analysis of Microemulsion

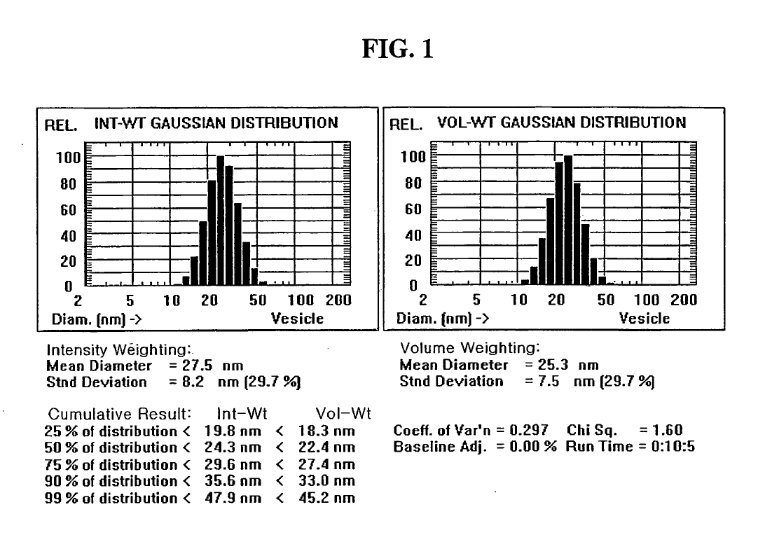

After diluting microemulsion preconcentrate manufactured in Example 1-a with water, the granularity distribution of the formed microemulsion was analyzed using a Nicomp 380. The results are shown in FIG. 1.

As is evident from FIG. 1, the microemulsion preconcentrate according to the present invention forms an oil-in-water microemulsion having an average particle diameter of 30 nm or less in the internal oil phase.

### EXPERIMENTAL EXAMPLE 2

### Study on Soft Capsule Deformation

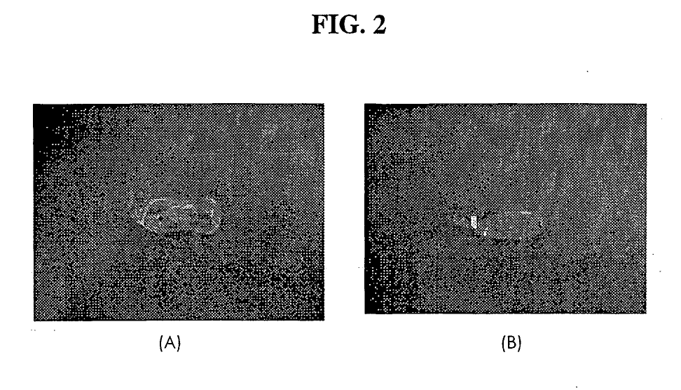

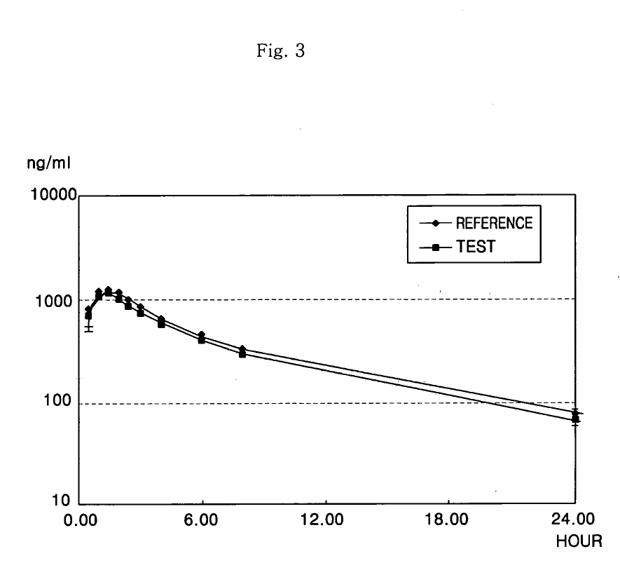

Changes in the shape of soft capsules were observed using the microemulsion preconcentrate prepared in Example 1-a and the conventional microemulsion preconcentrate prepared according to Example 3 of Korean Patent No. 01-31064. After filling empty soft capsules with each of the microemulsion preconcentrates, the soft capsules were left exposed to the air for 30 days before observation of the capsule appearance. As shown in the photographs of the soft capsules of FIG. 2, capsule B filled with the microemulsion preconcentrate according to the present invention retains its original shape perfectly, whereas capsule A filled with the conventional microemulsion preconcentrate is deformed due to interaction with the gelatin capsule shell.

### EXPERIMENTAL EXAMPLE 3

### Bioequivalence Test

A bioequivalence test was performed on 6 dogs using cyclosporin-containing microemulsion soft capsules (test capsules) prepared in Example 1, each capsule containing 100 mg of cyclosporin, and using Sandimmun Neoral of Novartis, reference capsules for comparison. The bioequivalence test was performed according to a 2×2 crossover study design using latin square method.

TABLE 5

Phase

| Group | Subject | I | II |
|---|---|---|---|
| 1 | A, B, C | Reference | Test |
| 2 | D, E. F | Test | Reference |

The six dogs were randomly divided into two groups of 3, and were labeled in alphabetical order. The above soft capsule containing 100 mg of cyclosporin was orally administered to each dog. Between the two treatments, one-week washout period is proveded. The test animals were no longer fed starting at noon, the day before the test day. In the test day, test and reference capsules were orally administered to the animals on an empty stomach, and no food or water was supplied for 4 hours following administration. The animals were fed 4 hours after administration.

2 ml of venous blood was collected using heparin-treated syringes from the cephatic vein at 0.5, 1.0, 1.5, 2.0, 2.5, 3.0, 3.5, 4.0, 6.0, 8.0, and 12.0 hours after administration of the test and reference capsules. The collected blood samples were immediately frozen at −60° C. The blood cyclosporin concentration was measured using radioimmuno assay (RIA). The results are shown in FIG. 3. Paracokinetic parameters of the test and reference capsules were calculated based on the data of FIG. 3. The results are shown in Table 6.

TABLE 6
Pharmacokinetic parameters of test and reference capsules

| | AUC(hr · ng/ml) | Cₘₐₓ (ng/ml) | Tₘₐₓ (hrs) |
|---|---|---|---|
| Reference | 8290.6 | 1241.1 | 1.33 |
| Test | 7649.7 | 1189.9 | 1.25 |
| % Deviation | −7.73% | −4.13% | −6.25% |

As is evident from the Experimental Examples, the microemulsion preconcentrate according to the present invention forms a stable microemulsion with an inner phase particle size of 30 nm or less, and has low reactivity with a gelatin soft capsule shell. The microemulsion preconcentrate according to the present invention is able to carry hydrophilic and protein drugs as well as hydrophobic drugs, poorly soluble in water, and ensures storage stability of the formulation because it does not interact with a gelatin capsule shell, during formulation.

1. A microemulsion preconcentrate comprising:
an active component;
an oil;
a surfactant; and
a hydrophilic solvent selected from the group consisting of propylene glycol diacetate, propylene glycol monoacetate, and salts of the forgoing materials.

2. The microemulsion preconcentrate of claim 1, wherein the ratio by weight of the sum of oil, hydrophilic solvent, and surfactant to the active component is 0.5-10.

3. The microemulsion preconcentrate of claim 1, wherein the ratio by weight of oil, hydrophilic solvent, and surfactant is 0.5-60: 0.5-60:0.5-80.

4. The microemulsion preconcentrate of claim 1, wherein the active component is selected from the group consisting of piroxicam, ketorolac, ketopropen, acetaminophen, aceclofenac, naproxen, gabapentin, amlodipine, felodipine, enalapril, isosorbide dinitrate, terazocine, carvedilol, nifedipine, captopril, itraconazole, fluconazole, ketoconazole, fluorouracil, paclitaxel, adriamycin, estradiol, progestin, testosterone, alprostadil, donepezil, rivastigmine, physostigmine, adrenol™, alendronate, cyclosporin, tacrolimus, ondansetron, scopolamine, meclizine, fluoxetine, venlafaxine, and pharmaceutically acceptable salts of the forgoing components.

5. The microemulsion preconcentrate of claim 1, wherein the active component is cyclosporin.

6. An oral pharmaceutical preparation comprising:
a microemulsion preconcentrate including:
an active component;
an oil;
a surfactant; and
a hydrophilic solvent selected from the group consisting of propylene glycol diacetate, propylene glycol monoacetate, and salts of the forgoing materials.

7. The oral pharmaceutical preparation of claim 6, wherein the oral pharmaceutical preparation is soft capsule, gelatin-sealed hard capsule, or liquid.

8. The oral pharmaceutical preparation of claim 6, wherein the ratio by weight of the sum of oil, hydrophilic solvent, and surfactant to the active component is 0.5-10.

9. The oral pharmaceutical preparation of claim 6, wherein the ratio by weight of oil, hydrophilic solvent, and surfactant is 0.5-60: 0.5-60:0.5-80.

10. The oral pharmaceutical preparation of claim 6, wherein the active component is selected from the group consisting of piroxicam, ketorolac, ketopropen, acetaminophen, aceclofenac, naproxen, gabapentin, amlodipine, felodipine, enalapril, isosorbide dinitrate, terazocine, carvedilol, nifedipine, captopril, itraconazole, fluconazole, ketoconazole, fluorouracil, paclitaxel, adriamycin, estradiol, progestin, testosterone, alprostadil, donepezil, rivastigmine, physostigmine, adrenol™, alendronate, cyclosporin, tacrolimus, ondansetron, scopolamine, meclizine, fluoxetine, venlafaxine, and pharmaceutically acceptable salts of the forgoing components.

11. The oral pharmaceutical preparation of claim 1, wherein the active component is cyclosporin.
